(19) United States
(12) Patent Application Publication
(10) Pub. No.: US 2026/0272650 A1
(43) Pub. Date: Sep. 17, 2026

(54) CARDIAC IMPLANT DEVICE

(71) Applicant: United InnoMed (Shanghai) Limited, Shanghai (CN)

(72) Inventors: Bo LI, Shanghai (CN); Li Wang, Shanghai (CN)

(73) Assignee: United InnoMed (Shanghai) Limited, Shanghai (CN)

(21) Appl. No.: 19/167,068

(22) PCT Filed: Mar. 22, 2024
(86) PCT No.: PCT/CN2024/083196

§ 371 Date: Sep. 19, 2025

(30) Foreign Application Priority Data
Mar. 24, 2023 (CN) 202310306016.4

(51) Int. Cl. A61F 2/24

(52) U.S. Cl. CPC A61F 2/2442

(57) ABSTRACT

Provided in the present disclosure is a cardiac implant device. The cardiac implant device comprises: a fixation member, which has a collapsed state in which the fixation member can pass through a created opening in the atrial septum, and an expanded state in which a pair of securing portions formed on two sides of the atrial septum and clamp the atrial septum secure the fixation member to the atrial septum; and a shunt member, which is configured for attaching to the fixation member and passing through the created opening, and is configured to define a shunt channel that passes through the created opening and is in communication with the left atrium and the right atrium of a patient. The fixation member can be reliably secured to the atrial septum, and thus the shunt member attached to the fixation member can be reliably secured to the atrial septum. In addition, since the fixation member has a collapsed state in which the fixation member can pass through the created opening in the atrial septum, during a delivery process, the fixation member can be kept in the collapsed state, so that the fixation member can smoothly pass through a narrow portion in a delivery path and partially pass through the created opening, thereby making the implant process of the shunt member easier.

This application claims the priority of the Chinese patent application CN202310306016.4 filed on Mar. 24, 2023. This application cites the entire text of the aforementioned Chinese patent application.

### TECHNICAL FIELD

The present disclosure relates to the technical field of medical devices, and in particular, to a cardiac implant device.

### BACKGROUND

Increased left atrial pressure, or even persistent high pressure, is an important cause of left heart failure. For heart failure patients with persistent elevated left atrial pressure at rest, or with significantly increased left atrial pressure and relatively low right atrial pressure during exercise, blood can be shunted from the left atrium to the right atrium by creating a created opening in the atrial septum and implanting a shunt device, thereby effectively reducing left atrial pressure and alleviating the patient's heart failure symptoms. However, with traditional shunt devices, after implantation, it is usually difficult to reliably secure the device to the atrial septum of the patient's atrium, and accidental and unfavorable change may occur to its position and posture. In addition, for traditional shunt devices, the process of implantation into the patient's heart is relatively difficult.

### SUMMARY

In view of these, the present disclosure provides a cardiac implant device to at least partially solve the problems that a traditional shunt device implantation process is relatively difficult and that it is difficult to reliably secure the device to the patient's atrial septum.

The cardiac implant device provided by the present disclosure comprises: a fixation member having a collapsed state for being capable of passing through a created opening in the atrial septum, and an expanded state in which the fixation member is secured to the atrial septum; and a shunt member configured to be attached to the fixation member and capable of passing through the created opening. The shunt member is configured to define a shunt channel that passes through the created opening and connects the patient's left atrium and right atrium.

In one possible embodiment, the fixation member comprises a pair of fixing parts capable of being secured to the atrial septum.

During the delivery of the fixation member from outside the body to the created opening in the atrial septum, the fixation member can be maintained in a collapsed state to facilitate smooth passage through narrow places along the delivery path. The collapsed fixation member can then be partially passed through the created opening, with one portion positioned proximal to the atrial septum (e.g., on the left side) and the other portion positioned distal to the atrial septum (e.g., on the right side). The fixation member can then be released, transitioning from the collapsed state to an expanded state, thereby forming a pair of fixing parts, one on each side of the atrial septum, that clamp onto the atrial septum. The shunt member can then be partially passed through the created opening to create the shunt channel connecting the left and right atria, and the shunt member can be attached to the fixation member. Of course, in other embodiments, the shunt member and the fixation member can be delivered to the created opening as a single unit.

According to the cardiac implant device provided by the present disclosure, the fixation member can be securely fixed to the atrial septum by a pair of fixing parts clamped to the atrial septum, thereby securely fixing the shunt member attached to the fixation member to the created opening in the atrial septum. Furthermore, because the fixation member has a collapsed state for being capable of passing through the created opening in the atrial septum, the fixation member can be maintained in the collapsed state during delivery, allowing it to smoothly pass through narrow places along the delivery path and partially pass through the created opening, thereby facilitating the shunt member implantation process.

In one possible embodiment, each fixing part is a fixing disc, and the shunt channel has a first opening and a second opening configured to respectively connect to the left atrium and the right atrium. The first opening and the second opening are configured as follows: a distance between the orthographic projections of the first opening and the second opening on a projection plane substantially parallel to the pair of fixing parts is in a range of 10 mm to 30 mm, so that when the patient is in at least one posture, the first opening is higher than the second opening in the direction of gravity.

With this configuration, when the patient assumes one or more specific postures, the first opening is higher than the second opening in the direction of gravity. As a result, blood and/or suspended particles (such as microthrombi) in the right atrium need to overcome gravity in order to pass through the shunt channel to the left atrium. This embodiment therefore helps prevent blood reflux (blood flowing from the right atrium to the left atrium). Blood flowing from the right atrium to the left atrium may cause health problems. For example, if thrombi or other particles in venous blood are not filtered by the pulmonary capillaries and directly enter the left atrium through the created opening in the atrial septum, and then enter the systemic arterial circulation (including the cerebral circulation) through the left atrium, the patient may suffer from problems such as cerebral stroke, sub-stroke, or blockage of small blood vessels in organ arteries.

In a possible embodiment, the first opening and the second opening are configured in such a way that an orthographic projection of the first opening on the projection plane is located further anterior and/or further superior than an orthographic projection of the second opening on the projection plane.

If the projection of the first opening on the projection plane is further anterior than the projection of the second opening on the projection plane (i.e., closer to the front side of the patient's body), then when the patient is in a supine posture (or a posture close thereto), the first opening will be higher than the second opening in the direction of gravity, thereby reducing the risk of blood reflux in this situation.

If the projection of the first opening on the projection plane is higher than the projection of the second opening on the projection plane (i.e., closer to the side of the patient's head), then when the patient is in an upright posture (or a posture close thereto, such as a sitting posture with the upper body upright), the first opening is higher than the second opening in the direction of gravity, thereby reducing the risk of blood reflux in this situation.

If the projection of the first opening on the projection plane is further anterior and further superior than the projection of the second opening on the projection plane, then when the patient is in an upright or supine position (or a position close to the upright or supine position), the first opening is higher than the second opening in the direction of gravity, thereby reducing the risk of blood reflux in such situations. Considering that people spend most of their time in an upright or supine posture (or a posture close to the upright or supine posture) in daily life, this configuration can better meet the needs of patients in daily activities.

In a possible embodiment, the shunt member is configured to be in a straight cylindrical shape and inclined relative to the projection plane.

This structure facilitates to increase the distance on the projection plane between the first opening and the second opening respectively located at two ends of the shunt member, so that the distance between the two meets the requirements (i.e., the distance between the first opening and the second opening on the projection plane in the range of 10 mm to 30 mm).

In one possible embodiment, the shunt member is configured to be substantially S-shaped.

Specifically, the shunt member may comprise a first portion, a second portion and a third portion, and the fixation member may comprise a first fixing disc and a second fixing disc. The first portion may be located on a side of the first fixing disc facing away from the second fixing disc, and the first portion extends approximately parallel to the first fixing disc. The second portion may be located on a side of the second fixing disc facing away from the first fixing disc, and the second part extends approximately parallel to the second fixing disc. The third portion is inserted into the fixation member, and its two ends are respectively connected to the first portion and the second portion. The first opening is provided on the first portion, in particular, the first opening may be provided on the end of the first portion. The second opening is provided on the second portion, in particular, the second opening may be provided on the end of the second portion.

Since the shunt member is constructed to be roughly S-shaped, after it is installed on the fixation member, its portions extending into the left and right atria will be respectively close to the pair of fixing discs, which helps to reduce the disturbance for blood flow caused by the portions of the shunt member extending into the left and right atria, thereby helping to avoid turbulence and reduce the formation of blood clots.

In one possible embodiment, the cardiac implant device comprises a plurality of shunt members.

After the shunt device is implanted, patients often receive other treatments, such as taking medication. After a period of time, some patients' conditions may improve. In this case, the shunt device may no longer be suitable for the patient's condition. Shunt channels with smaller cross-sectional areas are more likely to cause tissue growth than those with larger cross-sectional areas. The cardiac implant device provided herein comprises multiple shunt members, thus having multiple shunt channels. As a result, while the sum of the cross-sectional areas of the multiple shunt channels matches the patient's condition, the cross-sectional area of each shunt channel can be relatively small, making tissue growth more likely inside each channel. When the shunt device is first implanted in the patient's heart, the sum of the cross-sectional areas of the multiple shunt channels meets the patient's current condition at that time. After a period of usage, the patient's condition improves, and each shunt channel experiences a certain degree of tissue growth. Consequently, the sum of the cross-sectional areas of the multiple shunt channels decreases fairly rapidly, ultimately better matching the patient's current condition. In contrast, if a cardiac implant device only comprises one shunt member, the cross-sectional area of the shunt member's flow channel will be larger, making tissue growth less likely to occur inside the shunt, or the rate of tissue growth will be slower. Consequently, after a period of usage, the cardiac implant device may no longer be suitable for the patient's condition.

In a possible embodiment, the plurality of shunt members comprises a first shunt member and a second shunt member, and a cross-sectional area of the shunt channel of the first shunt member is greater than a cross-sectional area of the shunt channel of the second shunt member.

The first shunt member's shunt channel has a larger cross-sectional area. Therefore, after a period of use, there is a small amount of tissue growth in the shunt channel, maintaining connectivity between the left and right atria. Since the second shunt member's shunt channel has a smaller cross-sectional area, there is a large amount of tissue growth in the shunt channel after a period of usage, causing the shunt channel to become narrower or completely blocked, thereby reducing the overall shunt effect of the cardiac implant device. Therefore, this configuration facilitates better adaptation to the patient's evolving condition.

In a possible embodiment, the cross-sectional area of the shunt channel of the second shunt member is less than or equal to 7 mm². It has been proven that if the cross-sectional area of the shunt channel is less than or equal to 7 mm², after a period of usage, the shunt channel will be blocked due to tissue growth and other reasons, thereby reducing the overall shunt effect of the cardiac implant device, and better matching the development of the patient's condition.

In one possible embodiment, the plurality of shunt members comprises a first shunt member and a second shunt member, and the shunt channel of the second shunt member is configured to be more likely to cause tissue growth than the shunt channel of the first shunt member.

Because the second shunt member's shunt channel is configured to be more likely to cause tissue growth than the first shunt member's, after a period of usage, the first shunt member's shunt channel experiences less tissue growth, maintaining connectivity between the left and right atria. However, the second shunt member's shunt channel experiences more tissue growth, becoming narrower or completely blocked, thereby reducing the overall shunt effect of the cardiac implant device. This configuration therefore facilitates better adaptation to the patient's evolving condition.

In one possible embodiment, the inner wall of the shunt channel of the second shunt member is rougher than the inner wall of the shunt channel of the first shunt member. In this way, the shunt channel of the second shunt member is more likely to cause tissue growth than the shunt channel of the first shunt member.

In one possible embodiment, the inner wall of the shunt channel of the second shunt member is provided with a plurality of micropores to cause tissue growth. In this way, the shunt channel of the second shunt member is more likely to cause tissue growth than the shunt channel of the first shunt member.

In one possible embodiment, the plurality of micropores is filled with a filler that is degradable and can cause tissue growth. In this way, the shunt channel of the second shunt member is more likely to cause tissue growth than the shunt channel of the first shunt member.

In one possible embodiment, the inner wall of the shunt channel of the second shunt member is provided with a coating that is degradable and can cause tissue growth. In this way, the shunt channel of the second shunt member is more likely to cause tissue growth than the shunt channel of the first shunt member.

In one possible embodiment, the shunt member comprises a stent and a covering membrane located on the inner circumferential side or outer circumferential side of the stent. The stent comprises a pair of flared portions and a neck portion located therebetween, wherein the pair of flared portions are configured to be self-expanding and the neck portion is configured to be expandable.

The fixation member may be provided with a mounting hole for mounting the stent. During the process of mounting the stent to the fixation member, the pair of flared portions may be maintained in a collapsed state, and the stent may be inserted into the mounting hole. Subsequently, the pair of flared portions may be unfolded so that the pair of flared portions are located on opposite sides of the fixation member, respectively. Subsequently, the neck portion may be expanded by an operation device (e.g., a balloon) so that the neck portion matches the size of the mounting hole. According to this embodiment, the stent can be relatively conveniently and reliably fixed to the fixation member. In addition, since the neck portion is constructed to be expandable, during the implantation process, the inner diameter of the stent may be adjusted by an operation device (e.g., a balloon), thereby adjusting the inner diameter of the shunt channel (i.e., the inner diameter of the shunt channel at the neck portion), thereby making the inner diameter of the shunt channel better match the patient's condition.

In one possible embodiment, the shunt member comprises a stent and a covering membrane positioned on the inner circumferential side or outer circumferential side of the stent, wherein the cross-section of the stent has a shape of an open ring. This allows the inner diameter of the stent, and thus the inner diameter of the shunt channel, to be adjusted during implantation using an operation device (e.g., a balloon).

In a possible embodiment, the stent is provided with a plurality of first gripping elements on its outer circumferential side, and the stent is configured to be attached to the fixation member via the plurality of first gripping elements. The stent can be reliably fixed to the fixation member via the plurality of first gripping elements.

In one possible embodiment, the cardiac implant device further comprises a sensor configured to be attached to the fixation member to sense physiological information of the patient.

Compared to traditional shunt devices, the disclosed cardiac implant device not only achieves therapeutic purposes but also senses the patient's physiological information, thereby monitoring the heart's working condition and helping physicians diagnose and treat heart diseases and disease changes. Furthermore, the disclosed cardiac implant device reliably secures both the shunt member and the sensor to the created opening of the atrial septum using only a single fixation member. This eliminates the need for two fixation members in the patient's heart to secure the shunt member and the sensor, respectively, reducing the space occupied by the implant in the patient's heart and minimizing the damage caused by installing an additional fixation member on the atrial septum.

In one possible embodiment, the fixation member comprises a pair of fixing parts that can be fixed to the atrial septum and a waist part located between the pair of fixing parts and constructed to pass through the created opening, wherein one of the shunt member and the sensor passes through the waist part or is connected to the waist part, and the other does not pass through the waist part. Each fixing part is a fixing disc.

In this embodiment, the waist part of the fixation member can maintain the distance between the shunt member and the sensor, preventing the two from being too close to each other. If the shunt member and the sensor are too close, the shunt member may affect the accuracy of the detection result of the sensor.

In one possible embodiment, the fixation member comprises a pair of fixing parts capable of being secured to the atrial septum, and a waist part located between the pair of fixing parts and configured to pass through the created opening. Both the shunt member and the sensor pass through the waist part. This configuration facilitates better fit of the entire cardiac implant device within the created opening in the atrial septum. Each fixing part is a fixing disc.

In one possible embodiment, each fixing part is elliptical with a pair of focal points, with the shunt member and sensor located at each of the focal points. This ensures that the cardiac implant device fits snugly within the created opening in the atrial septum while maintaining a sufficient distance between the shunt member and the sensor.

In one possible embodiment, the sensor is configured to pass through the created opening, and at least one end of the sensor is provided with a sensing unit that is at least partially exposed outside the fixation member.

Since the sensor is constructed to pass through the created opening, the two ends of the sensor are respectively located in the left and right atria, so that the two ends of the sensor are closer to the atrial septum. This structure helps to reduce the disturbance for the blood flow in the left and right atria by the sensor, thereby helping to avoid turbulence and reduce the formation of blood clots. The sensing unit is exposed outside the fixation member, that is, the sensing unit is directly exposed to the blood flow, which helps to improve the accuracy of the sensing results. In addition, in the embodiment wherein the sensing units are provided at both ends of the sensor, the sensor can detect the physiological information of the left and right atria at the same time. In this way, more accurate patient's health status can be obtained from the subsequent analysis based on the sensing results.

In one possible embodiment, the fixation member comprises a pair of fixing parts that can be fixed to the atrial septum. The fixing parts are fixing discs, and the pair of fixing discs comprise a first fixing disc and a second fixing disc, wherein the first fixing disc is fixed with a retaining ring, and the sensor is fixed to the second fixing disc and is provided with one or more clips adapted to the retaining ring.

During implantation, after the fixation member is deployed, the first and second fixing discs are formed respectively on two sides of the atrial septum. The sensor can then be moved from the second fixing disc toward the first fixing disc, making one or more of the sensor's clips to engage with the retaining ring. The pair of fixing discs are compressed to clamp the patient's atrial septum. In particular, in embodiments where the sensor is equipped with multiple clips, some clips can be selected from the multiple clips to engage with the retaining ring, to adjust how tightly the pair of fixing discs clamp the atrial septum, to accommodate thickness of the atrial septum among different patients.

In one possible embodiment, at least a portion of the retaining ring is made of a radiopaque material.

During implantation, the sensor can be connected to a delivery tool, allowing the physician to determine the sensor's position. Because at least a portion of the retaining ring is made of a radiopaque material, the physician can also determine the retaining ring's position during implantation. This allows the physician to accurately move the sensor based on the positions of the sensor and the retaining ring to properly engage the retaining ring with the clips. In particular, in embodiments where the sensor is provided with multiple clips, the physician can determine which clips the retaining ring engages based on positions of the sensor and the retaining ring, thereby determining how tightly the atrial septum is clamped by the pair of fixing discs.

In one possible embodiment, the sensor further comprises a communication unit for communicating with an external device, wherein the one or more clips at least partially offset from the communication unit in a longitudinal direction of the sensor. With this configuration, the one or more clips will have no or minimal impact on the quality of communication between the communication unit and the external device.

In one possible embodiment, a plurality of second gripping elements is provided on the outer surface of the sensor, and the sensor is attached to the fixation member via the second gripping elements. The sensor can be reliably fixed to the fixation member via the plurality of second gripping elements.

In one possible embodiment, each second gripping element has an open-ring shape, which helps reduce the overall weight and size of the multiple gripping elements, thereby facilitating miniaturization and lightweight of the cardiac implant device.

In a possible embodiment, each second gripping element has a closed-ring shape. According to this structure, the sensor can be more reliably fixed on the fixation member.

In one possible embodiment, the plurality of second gripping elements comprise two groups of second gripping elements spaced apart in the longitudinal direction of the sensor, and the two groups of second gripping elements are bent toward each other. According to this configuration, the sensor can be more reliably fixed to the fixation member.

In one possible embodiment, the sensor comprises a cylindrical main body and a sleeve sleeved on the main body, wherein the plurality of second gripping elements is attached to the main body via the sleeve. In this way, the plurality of second gripping elements can be fixed to the main body of the sensor.

In one possible embodiment, the sleeve is configured to be slidable longitudinally along the main body. This allows the physician to adjust the position of the sensor's main body during implantation based on practical circumstances. For example, the main body can be adjusted to ensure that the sensing unit is directly exposed to the blood in the atrium, or to ensure that the communication unit's signal is not excessively obstructed.

In one possible embodiment, the sensor further comprises a communication unit for communicating with an external device, wherein the plurality of second gripping elements and the communication unit do not overlap in the longitudinal direction of the sensor. With this configuration, the plurality of second gripping elements will have little or no impact on the communication quality between the communication unit and the external device.

In one possible embodiment, the fixation member comprises a pair of fixing parts capable of being fixed to the atrial septum. The sensor further comprises a communication unit for communicating with an external device, wherein the sensing unit is configured to be tilted relative to the pair of fixing parts so that the communication unit is away from the shunt member; and each fixing part is a fixing disc. With this configuration, the shunt member will have no or minimal impact on the communication quality between the communication unit and the external device.

In one possible embodiment, the sensor is configured to be located between the shunt member and the patient's anterior chest wall, or alternatively, between the shunt member and the patient's left chest wall; or alternatively, between the shunt member and the patient's posterior chest wall. With this configuration, the shunt member will have no or minimal impact on the communication quality between the sensor's communication unit and the external device.

In one possible embodiment, the fixation member comprises a pair of fixing parts capable of being fixed to the atrial septum; and the fixing parts are arranged in an offset manner, and the shunt member is configured to be attached to one of the fixing parts. With this configuration, the overall size of the fixation member can be relatively small while ensuring reliable fixation of the cardiac implant device to the atrial septum. Each fixing part is a fixing disc.

In one possible embodiment, the fixation member comprises a pair of fixing parts capable of being fixed to the atrial septum. The pair of fixing parts comprises a first fixing disc and a second fixing disc, the first fixing disc being larger than the second fixing disc, and the shunt member is attached to the first fixing disc. With this configuration, the overall size of the fixation member can be relatively small while ensuring reliable fixation of the cardiac implant device to the atrial septum.

In one possible embodiment, the fixation member comprises a pair of fixing parts capable of being fixed to the atrial septum. The fixation member is radially woven from strands of wire such that the braiding density of each fixing part gradually decreases from the position where the sensor is located to the edge of each fixing part. This configuration helps reduce the adverse effects of the fixation member on the communication quality between the communication unit and external devices.

### BRIEF DESCRIPTION OF THE DRAWINGS

In order to more clearly illustrate the technical solutions of the embodiments of the present disclosure, the following briefly introduces the drawings used in the embodiments.

It should be understood that the following drawings only illustrate certain embodiments of the present disclosure and should not be considered limiting of the scope.

It should also be understood that the same or similar reference numerals are used in the drawings to identify the same or similar elements.

It should also be understood that the drawings are merely schematic and that the sizes and proportions of elements in the drawings are not necessarily accurate.

FIG. 1 is a schematic structural diagram of a cardiac implant device according to an embodiment of the present disclosure.

FIG. 2 is a schematic diagram showing projections of a pair of openings of a shunt channel of a shunt member of the cardiac implant device in FIG. 1 on a projection plane.

FIG. 3 is a schematic structural diagram of a cardiac implant device according to another embodiment of the present disclosure.

FIG. 4 is a schematic structural diagram of a cardiac implant device according to another embodiment of the present disclosure.

FIG. 5 is a schematic structural diagram of a cardiac implant device according to another embodiment of the present disclosure.

FIG. 6 is a schematic structural diagram showing the cardiac implant device shown in FIG. 5 viewed from another perspective.

FIG. 7 is a schematic structural diagram of a cardiac implant device according to another embodiment of the present disclosure.

FIG. 8a and FIG. 8b are schematic structural diagrams of a cardiac implant device according to another embodiment of the present disclosure.

FIG. 9 is a schematic structural diagram of a cardiac implant device according to another embodiment of the present disclosure.

FIG. 10 is a schematic structural diagram of a cardiac implant device according to another embodiment of the present disclosure.

FIG. 11 is a schematic structural diagram of a cardiac implant device according to another embodiment of the present disclosure.

FIG. 12 is a schematic structural diagram of a cardiac implant device according to another embodiment of the present disclosure.

FIG. 13 is a schematic structural diagram of a cardiac implant device according to another embodiment of the present disclosure.

FIG. 14 is a schematic structural diagram of a cardiac implant device according to another embodiment of the present disclosure.

FIG. 15 is a schematic structural diagram of a cardiac implant device according to another embodiment of the present disclosure.

FIG. 16 is a schematic structural diagram of a cardiac implant device according to another embodiment of the present disclosure.

FIG. 17 is a schematic structural diagram of a stent according to an embodiment of the present disclosure.

FIG. 18 is a schematic structural diagram of a cardiac implant device according to another embodiment of the present disclosure.

FIG. 19 is a schematic structural diagram of a cardiac implant device according to another embodiment of the present disclosure.

FIG. 20 is a schematic structural diagram of a cardiac implant device according to another embodiment of the present disclosure.

FIG. 21a and FIG. 21b are schematic structural diagrams of a stent according to another embodiment of the present disclosure.

FIG. 22 is a schematic structural diagram of a sensor according to an embodiment of the present disclosure.

FIG. 23 is a schematic structural diagram of a sensor according to another embodiment of the present disclosure.

### DESCRIPTION OF PREFERRED EMBODIMENTS

Followed is an exemplary description of the embodiments of the present disclosure in conjunction with the accompanying drawings. It should be understood that the present disclosure can be implemented in many ways and should not be construed as being limited to the embodiments described herein. The embodiments described herein are only for a more thorough and complete understanding of the present disclosure.

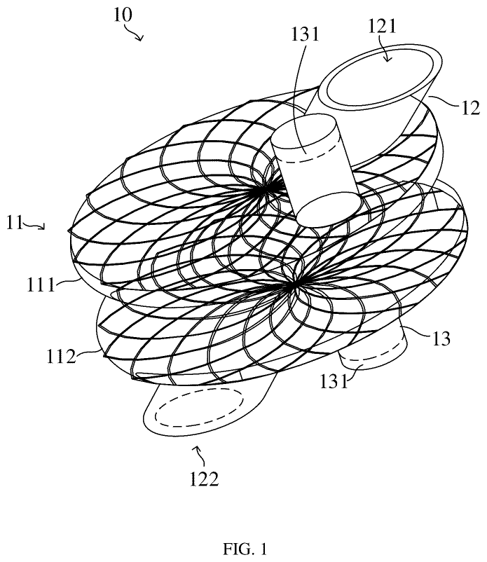

FIG. 1 shows a cardiac implant device 10 according to an embodiment of the present disclosure. Referring to FIG. 1, the cardiac implant device 10 comprises a fixation member 11 and a shunt member 12. The fixation member 11 has a collapsed state and an expanded state. In the collapsed state, the fixation member 11 can pass through the created opening in the atrial septum of a patient's atrium. In the expanded state, the fixation member 11 can form a pair of fixing discs 111, 112 (also referred to as fixing parts 111, 112) on two sides of the atrial septum and are clamped on the atrial septum to fix the fixation member 11 on the atrial septum. The pair of fixing discs 111, 112 are substantially parallel. The shunt member 12 is constructed to be attached to the fixation member 11 and to pass through the created opening in the atrial septum, and is constructed to define a shunt channel that passes through the created opening and connects the patient's left atrium and right atrium.

During the delivery of the fixation member 11 from outside the body to the created opening in the atrial septum, the fixation member 11 can be maintained in a collapsed state to facilitate smooth passage through narrow places along the delivery path. The collapsed fixation member 11 can then be partially passed through the created opening, with one portion of the fixation member 11 positioned proximal to the atrial septum (e.g., on the left side) and the other portion positioned distal to the atrial septum (e.g., on the right side). The fixation member 11 can then be released, transitioning from the collapsed state to the expanded state, thereby forming a pair of fixing discs 111, 112, positioned respectively on two sides of the atrial septum and clamped to the atrial septum. The shunt member 12 can then be partially passed through the created opening to create a shunt channel connecting the left and right atria, and the shunt member 12 can be attached to the fixation member 11. It should be noted that, in other embodiments, the shunt member 12 and the fixation member 11 can also be delivered to the created opening as a whole at one time. For example, the shunt member 12 and the fixation member 11 can be integrally formed. When the fixation member 11 changes from a collapsed state to an expanded state, so that a pair of fixing parts are located on two sides of the atrial septum, the shunt member 12 is simultaneously located at the created opening so that the shunt channel connects the left and right atria.

According to the cardiac implant device 10 provided in the present disclosure, the pair of fixing discs 111, 112 clamped to the atrial septum enable the fixation member 11 to be securely fixed to the atrial septum, thereby enabling the shunt member 12 attached to the fixation member 11 to be securely fixed to the atrial septum. Furthermore, because the fixation member 11 has a collapsed state for being capable of passing through the created opening in the atrial septum, the fixation member 11 can be maintained in the collapsed state during delivery, allowing it to smoothly pass through narrow areas of the delivery path and partially pass through the created opening, thereby facilitating the implantation of the shunt member 12.

In one embodiment, at least a portion of the fixation member 11 (e.g., the pair of fixing discs 111, 112) can be braided from elastic wires, allowing the fixation member 11 to have both a collapsed state and an expanded state. Of course, in other embodiments, the fixation member 11 can also be implemented in other ways. In another embodiment, both the fixation member 11 and the shunt member 12 are braided from elastic wires.

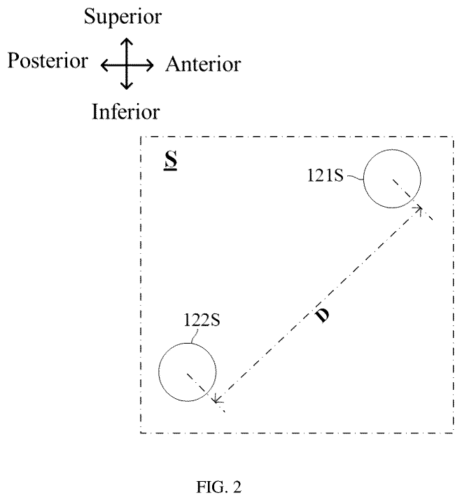

Continuing with FIG. 1, the shunt channel of the shunt member 12 has a first opening 121 and a second opening 122. The first opening 121 and the second opening 122 are configured to connect to the patient's left atrium and right atrium, respectively. FIG. 2 schematically illustrates orthographic projections of the pair of openings 121, 122 on a projection plane S. The projection plane S is substantially parallel to the pair of fixing discs 111, 112 (in the deployed state). For example, the angle between the projection plane S and the pair of fixing discs 111, 112 can be less than 10 degrees. It should be noted that when the cardiac implant device 10 is located in the patient's heart, the pair of fixing discs 111, 112 are substantially parallel to the patient's atrial septum. In this case, the plane on which the atrial septum lies can be considered as the projection plane S.

As shown in FIG. 2, the first opening 121 and the second opening 122 are configured such that, when the cardiac implant device 10 is located in the patient's heart, the distance D between the orthographic projections 121S and 122S of the first opening 121 and the second opening 122 on the projection plane S ranges from 10 mm to 30 mm. This allows the first opening 121 to be higher than the second opening 122 in the direction of gravity when the patient is in at least one posture. Specifically, the distance D between the orthographic projections 121S and 122S can range from 15 mm to 25 mm.

According to this configuration, when the patient assumes one or more certain postures, the first opening 121 will be higher than the second opening 122 in the direction of gravity. As a result, blood and/or suspended particles (such as microthrombi) in the right atrium need to overcome gravity in order to pass through the shunt channel to the left atrium. Therefore, this implementation helps prevent blood reflux (blood flowing from the right atrium to the left atrium). If blood flows from the right atrium to the left atrium, health problems may occur. For example, if thrombi or other particles in venous blood are not filtered by the pulmonary capillaries and directly enter the left atrium through the created opening of the atrial septum, and then enter the systemic arterial circulation (including the cerebral circulation) through the left atrium, the patient may suffer from problems such as cerebral stroke, sub-stroke, or blockage of small blood vessels in organ arteries.

Continue with FIG. 1 and FIG. 2, the first opening 121 and the second opening 122 are configured such that, when the cardiac implant device 10 is located in the patient's heart, an orthographic projection 121S of the first opening 121 on the projection plane S is located further anterior and/or further superior than an orthographic projection 122S of the second opening 122 on the projection plane S.

It should be noted that, in the embodiments of the present disclosure, the superior side in the up-down direction may refer to the side closer to the patient's head, the inferior side in the up-down direction may refer to the side closer to the patient's lower limbs, the anterior side in the front-to-back direction may refer to the side closer to the front of the patient's body, and the posterior side in the front-to-back direction may refer to the side closer to the rear of the patient's body.

If the projection 121S of the first opening 121 on the projection plane S is located further anterior (i.e., closer to the front of the patient's body) than the projection 122S of the second opening 122 on the projection plane S, then when the patient is in a supine position (or a position close thereto), the first opening 121 will be higher than the second opening 122 in the direction of gravity, thereby reducing the risk of blood reflux in this situation. If the projection 121S of the first opening 121 on the projection plane S is located further superior (i.e., closer to the patient's head) than the projection 122S of the second opening 122 on the projection plane S, then when the patient is in an upright posture (or a posture close thereto, such as a sitting posture with the upper body upright), the first opening 121 will be higher than the second opening 122 in the direction of gravity, thereby reducing the risk of blood reflux in this situation. If the projection 121S of the first opening 121 on the projection plane S is further anterior and further superior than the projection 122S of the second opening 122 on the projection plane S, then when the patient is in an upright or supine posture (or a posture close to the upright or supine position), the first opening 121 is higher than the second opening 122 in the direction of gravity, thereby reducing the risk of blood reflux in this situation. Considering that people spend most of their time in an upright or supine posture (or a posture close to the upright or supine posture) in daily life, this configuration can generally meet the needs of patients in daily activities.

Continuing with FIG. 1 and FIG. 2, the shunt member 12 can be constructed in a straight cylindrical shape and tilted relative to the projection plane S (or, in other words, relative to the pair of fixing discs 111, 112). In other words, the straight cylindrical shunt member 12 is not perpendicular to the projection plane S (or, in other words, to the pair of fixing discs 111, 112). This configuration facilitates increasing the distance D between the orthographic projections 121S and 122S on the projection plane S, of the first opening 121 and the second opening 122 respectively located at the ends of the shunt member 12, such that the distance D can range from 10 mm to 30 mm.

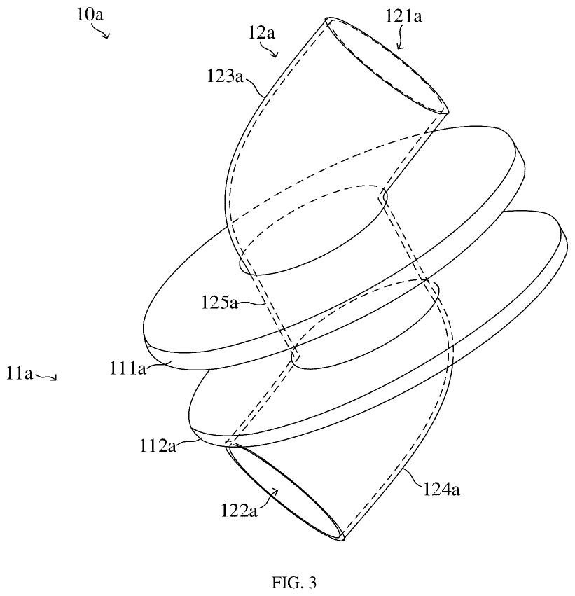

It should be understood that there are many ways to implement the shunt member, and the embodiments are not limited to those described above. Below, with reference to FIG. 3, an exemplary description of a shunt member 12a according to another embodiment of the present disclosure is provided. It should be noted that, in order to more clearly illustrate the shunt member 12a, the structure of the cardiac implant device 10a shown in FIG. 3 is simplified. For example, in certain embodiments, the cardiac implant device 10a may also comprise a sensor. Embodiments of such sensors will be provided below and will not be further elaborated here.

Referring to FIG. 3, the shunt member 12a is constructed substantially in an S-shaped configuration. Specifically, the shunt member 12a may comprise a first portion 123a, a second portion 124a, and a third portion 125a. The first portion 123a may be located on the side of the first fixing disc 111a facing away from the second fixing disc 112a, and may extend substantially parallel to the first fixing disc 111a (e.g., the angle therebetween may be less than 20 degrees). The second portion 124a may be located on the side of the second fixing disc 112a facing away from the first fixing disc 111a, and may extend substantially parallel to the second fixing disc 112a (e.g., the angle therebetween may be less than 20 degrees). The third portion 125a is inserted into the fixation member 11a, with its ends connected to the first portion 123a and the second portion 124a, respectively. The first opening 121a is provided on the first portion 123a, and in particular, may be provided at the end of the first portion 123a. The second opening 122a is provided on the second portion 124a. Specifically, the second opening 122a may be provided at the end of the second portion 124a.

Since the shunt member 12 a is constructed to be substantially S-shaped, the portions thereof extending into the left and right atria (i.e., the first portion 123a and the second portion 124a) will be respectively close to the pair of fixing discs 111a and 112a. This helps reduce the disturbance for blood flow by the portions of the shunt member 12a extending into the left and right atria (i.e., the first portion 123a and the second portion 124a), thereby helping to avoid turbulence and reduce the formation of blood clots.

Referring back to FIG. 1, the cardiac implant device 10a may further comprise a sensor 13. The sensor 13 is configured to be attached to the fixation member 11 and is used to sense physiological information of the patient. For example, the physiological information may comprise, but is not limited to, one or more of: blood pressure information in the patient's heart, pressure differences between different atria, blood flow rate information, blood pH information, blood temperature information, blood oxygen information, cardiac acoustic information, cardiac contractility information, cardiac acceleration information, cardiac chamber size and variation information, and intracardiac electrocardiogram information.

Compared to traditional shunt devices, the disclosed cardiac implant device 10 not only achieves therapeutic purposes but also senses the patient's physiological information, thereby monitoring the heart's working condition and assisting physicians in diagnosing and treating heart diseases and disease developments. Furthermore, the disclosed cardiac implant device 10 reliably secures both the shunt member 12 and the sensor 13 to the created opening of the atrial septum using only a single fixation member 11. This eliminates the need for two fixation members in the patient's heart to secure the shunt member and sensor separately. This reduces the space occupied by the implant in the patient's heart and minimizes the damage caused by installing an additional fixation member on the atrial septum.

Continuing with FIG. 1, the sensor 13 is configured to pass through the created opening in the atrial septum. The sensor 13 may comprise two sensing units 131 provided at two ends of the sensor 13, respectively, and each sensing unit 131 at least partially exposed outside the fixation member 11. It should be noted that in some embodiments, the sensor 13 may comprise only one sensing unit 131, located at one end of the sensor 13 and at least partially exposed outside the fixation member 11.

Since the sensor 13 is constructed to pass through the created opening, the two ends of the sensor 13 are respectively located in the left and right atria, which makes the two ends of the sensor 13 closer to the atrial septum. This structure is conducive to reducing the disturbance for the blood flow in the left and right atria by the sensor 13, thereby helping to avoid turbulence and reduce the formation of blood clots. In addition, the sensing unit 131 is exposed outside the fixation member 11, that is, the sensing unit 131 is directly exposed to the blood flow, which is conducive to improving the accuracy of the sensing results. In addition, in the embodiment method in which the sensing unit 131 is provided at both ends of the sensor 13, the sensor 13 can sense the physiological information of the left and right atria at the same time. In this way, more accurate patient's health status can be obtained by the subsequent analysis based on the sensing results can.

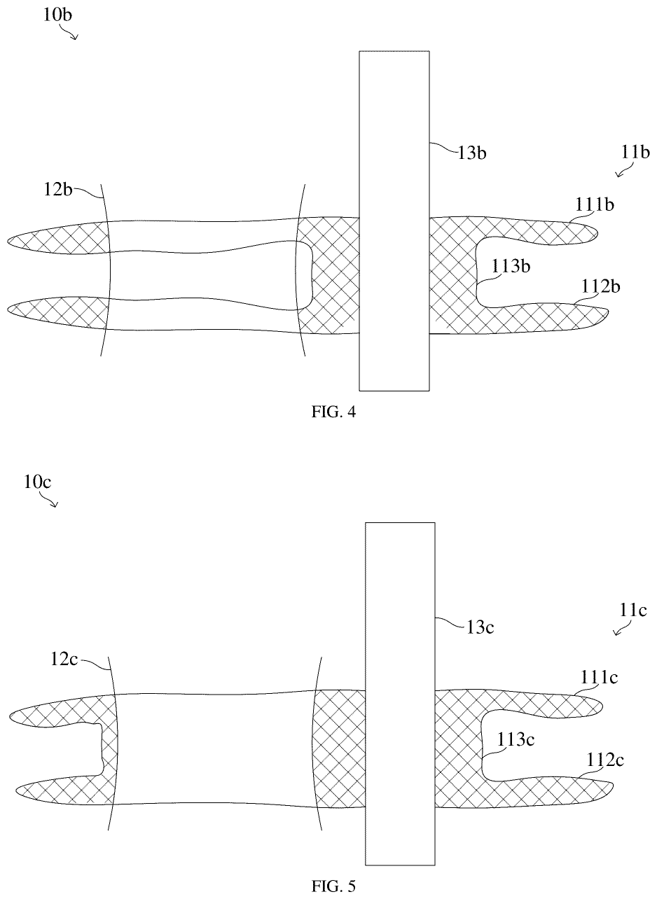

FIG. 4 illustrates a cardiac implant device 10b according to another embodiment of the present disclosure. Referring to FIG. 4, cardiac implant device 10b comprises a fixation member 11b, a shunt member 12b, and a sensor 13b. Fixation member 11b comprises a pair of fixing discs 111b, 112b (also referred to as fixing parts 111b, 112b), and a waist part 113b positioned between the pair of fixing discs 111b, 112b and configured to pass through a created opening in the atrial septum of a patient's atrium. Sensor 13b passes through waist part 113b, while shunt member 12b does not pass through waist part 113b. It should be noted that, in other embodiments, shunt member 12b may pass through waist part 113b, while sensor 13b does not pass through waist part 113b. In this case, the shunt member may also be integrally formed with fixation member 11b.

In this embodiment, the waist part 113b of the fixation member 11b can reliably maintain the distance between the shunt member 12b and the sensor 13b, preventing the two from being too close to each other. If the shunt member 12b and the sensor 13b are too close, the shunt member 12b may interfere with the accuracy of the sensing result of the sensor 13b.

FIG. 5 illustrates a cardiac implant device 10c according to another embodiment of the present disclosure. Referring to FIG. 5, cardiac implant device 10c comprises a fixation member 11c, a shunt member 12c, and a sensor 13c. Fixation member 11c comprises a pair of fixing discs 111c, 112c, and a waist part 113c positioned between the pair of fixing discs 111c, 112c and configured to pass through a created opening in the atrial septum of a patient's atrium. Both the shunt member 12c and the sensor 13c pass through waist part 113c. This configuration facilitates better integration of cardiac implant device 10c as a whole with the created opening in the atrial septum.

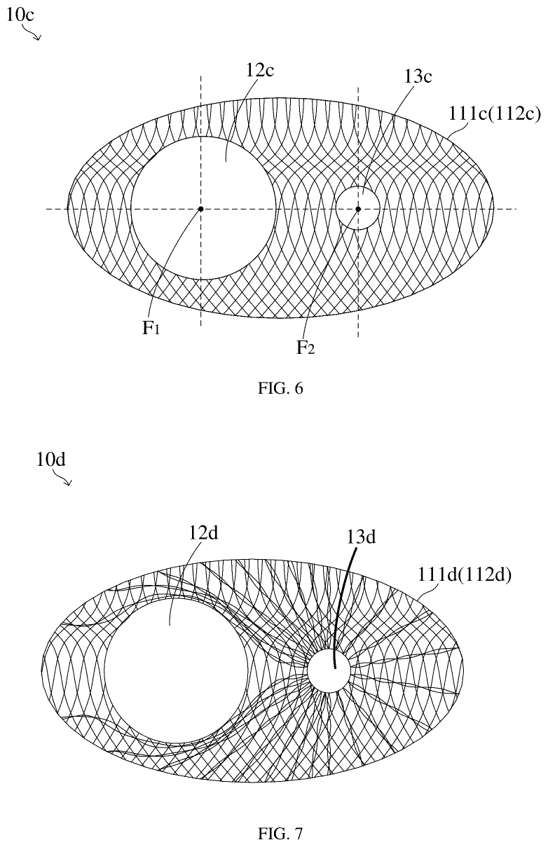

FIG. 6 shows a cardiac implant device 10c viewed from another direction. Referring to FIG. 6, each of the pair of fixing discs 111c, 112c is elliptical in shape, with a pair of focal points F1 and F2. The shunt member 12c and sensor 13c are located at these focal points, F1 and F2, respectively. This ensures that the entire cardiac implant device 10c fits well within the created opening in the atrial septum, while also maintaining a sufficient distance between the shunt member 12c and sensor 13c.

FIG. 7 illustrates a cardiac implant device 10d according to another embodiment of the present disclosure. Referring to FIG. 7, cardiac implant device 10d comprises a fixation member, a shunt member 12d, and a sensor 13d. The shunt member 12d and the sensor 13d are configured to be attached to the fixation member. The fixation member comprises a pair of fixing discs 111d, 112d. Each of the pair of fixing discs 111d, 112d is radially woven from strands of wire, such that the braiding density of each fixing disc gradually decreases from the location of the sensor 13d toward the edge of each fixing disc. This configuration helps reduce the adverse effects of the fixation member on the communication quality between the communication unit of the sensor 13d and an external device.

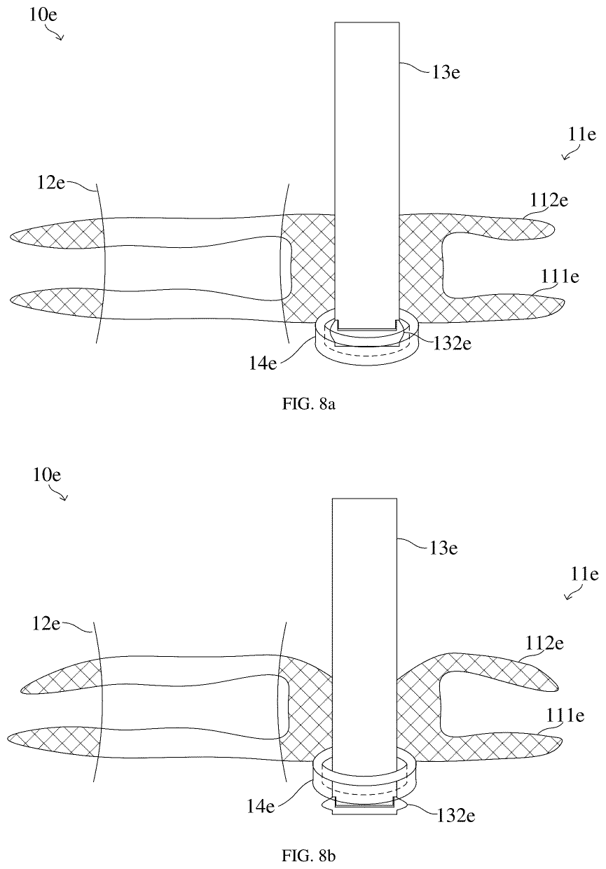

FIGS. 8a and 8b illustrate a cardiac implant device 10e according to another embodiment of the present disclosure. Referring to FIGS. 8a and 8b, cardiac implant device 10e comprises a fixation member 11e, a shunt member 12e, and a sensor 13e. The shunt member 12e and sensor 13e are attached to the fixation member 11e. The fixation member 11e comprises a pair of fixing discs 111e and 112e. The cardiac implant device 10e also comprises a retaining ring 14e, which is fixed to the first fixing disc 111e. The sensor 13e is fixed to the second fixing disc 112e and is provided with a clip 132e that mates with the retaining ring 14e.

During the implantation, after the fixation member 11e is deployed, a first fixing disc 111e and a second fixing disc 112e are formed on either side of the atrial septum. At this stage, the cardiac implant device 10e is in a state shown in FIG. 8a. Next, the sensor 13e can be moved from the second fixing disc 112e toward the first fixing disc 111e, so that the retaining ring 14e of the sensor 13 engages with the clip 132e. This presses the pair of fixing discs 111e and 112e to clamp the patient's atrial septum. At this stage, the cardiac implant device 10e is in a state shown in FIG. 8b.

As an exemplary embodiment, referring again to FIGS. 8a and 8b, clip 132e is elastic and thus compressible. Initially, as shown in FIG. 8a, clip 132e is located within and compressed in retaining ring 14e. After sensor 13e is moved from second fixing disc 112e toward first fixing disc 111e, clip 132e disengages from retaining ring 14e and expands. Thus, clip 132e cooperates with retaining ring 14e to securely clamp the pair of fixing discs 111e and 112e to the atrial septum of the patient's atrium.

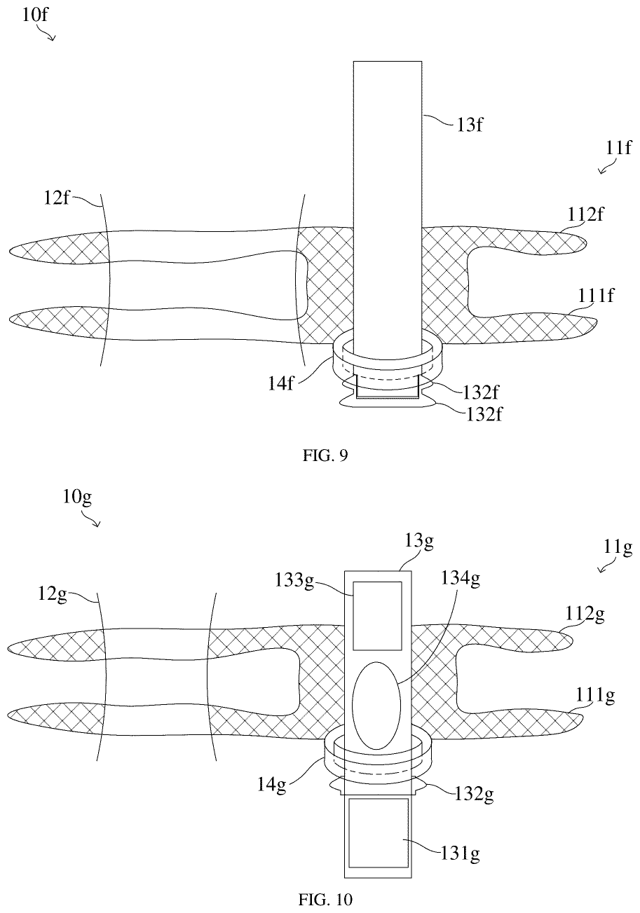

It should be noted that in the cardiac implant device provided herein, the sensor may be provided with only one clip or multiple clips along its longitudinal direction. The present disclosure does not specifically limit the number of clips in the fixation member. FIG. 9 illustrates a cardiac implant device 10f according to another embodiment of the present disclosure. Cardiac implant device 10f is substantially identical to cardiac implant device 10e. Cardiac implant device 10f comprises a fixation member 11f having a pair of fixing discs 111f and 112f, a shunt member 12f, a sensor 13f, and a retaining ring 14f. Fixation member 11f has multiple clips 132f (e.g., two clips 132f) arranged longitudinally thereof. Because fixation member 11f has multiple clips 132f, the degree of clamping of the pair of fixing discs 111f and 112f on the atrial septum can be adjusted by selecting a clip 132f from the multiple clips 132f that mates with the retaining ring 14f to accommodate the thickness of the atrial septum of different patients.

Referring again to FIGS. 8a and 8b, at least a portion of the retaining ring 14e can be made of a radiopaque material. For example, the radiopaque material can be, but is not limited to, one or more selected from platinum-iridium alloy, tantalum, or platinum. During implantation, the sensor 13e can be connected to a delivery tool, allowing the physician to determine the position of the sensor 13e. Because at least a portion of the retaining ring 14e is made of a radiopaque material, the physician can also determine the position of the retaining ring 14e during implantation. This allows the physician to accurately move the sensor 13e based on the positions of the sensor 13e and the retaining ring 14e, ensuring that the retaining ring 14e properly engages with the clip 132e. In particular, in embodiments where the sensor 13e is provided with multiple clips 132e, the physician can determine which clip 132e the retaining ring 14e engages based on the positions of the sensor 13e and the retaining ring 14e, thereby determining how tightly the atrial septum is clamped by the pair of fixing discs 111e and 112e. In another embodiment, the clip 132e may also be made of a radiopaque material to further facilitate the physician's judgment.

This disclosure does not specifically limit the location of the clips on the sensor. In one embodiment of this disclosure, the sensor may further comprise a communication unit for communicating with an external device, and one or more clips do not overlap with the communication unit and/or the sensing unit in the longitudinal direction of the sensor. With this configuration, the one or more clips will have little or no impact on the communication quality between the communication unit and the external device. This implementation is illustrated below with an embodiment.

FIG. 10 illustrates a cardiac implant device 10g according to another embodiment of the present disclosure. Referring to FIG. 10, cardiac implant device 10g comprises a fixation member 11g, a shunt member 12g, a sensor 13g, and a retaining ring 14g. The fixation member 11g comprises a pair of fixing discs 111g and 112g (also referred to as fixing parts 111g and 112g). The retaining ring 14g is secured to the first fixing disc 111g. The sensor 13g is secured to the second fixing disc 112g and is provided with a clip 132g that mates with the retaining ring 14g.

In addition to a sensing unit 131g, the sensor 13g also comprises a communication unit 133g. The communication unit 133g is used to communicate with an external device (i.e., a device located outside the patient's body). In one embodiment, the sensor 13g may also comprise a processing unit 134g, which can be used to process the physiological signals sensed by the sensing unit 131g. As shown in FIG. 10, in this embodiment, the clip 132g does not overlap with the sensing unit 131g and/or the communication unit 133g in the longitudinal direction of the sensor 13g.

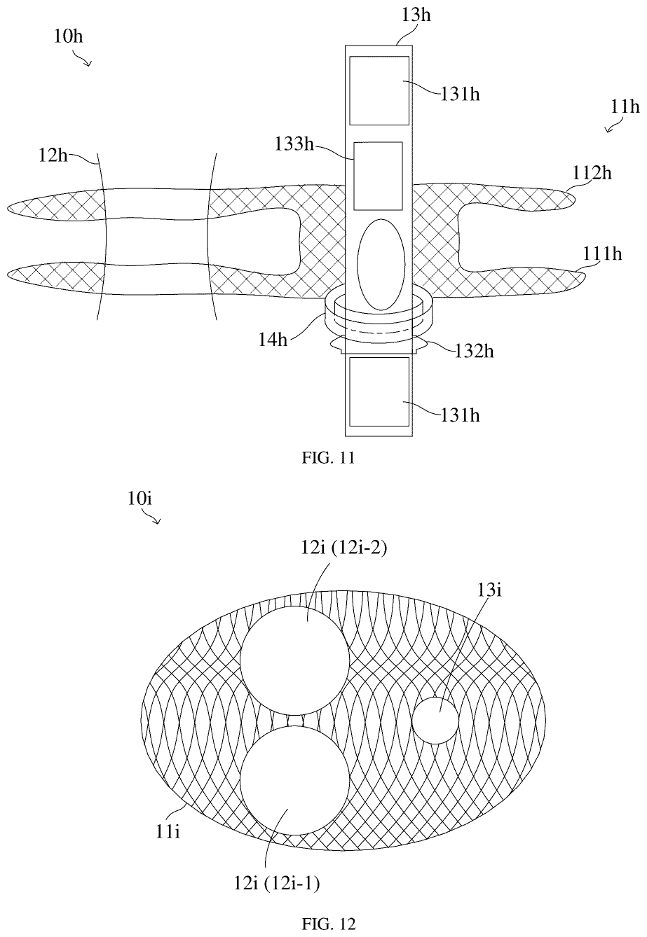

FIG. 11 illustrates a cardiac implant device 10h according to another embodiment of the present disclosure. Referring to FIG. 11, cardiac implant device 10h comprises a fixation member 11h, a shunt member 12h, a sensor 13h, and a retaining ring 14h. Fixation member 11h comprises a pair of fixing discs 111h and 112h. Retaining ring 14h is fixed to first fixing disc 111h. Sensor 13h is fixed to second fixing disc 112h and is provided with a clip 132h that mates with retaining ring 14h. Sensor 13h comprises a pair of sensing units 131h and a communication unit 133h. The pair of sensing units 131h are located at opposite ends of sensor 13h. As shown in FIG. 11, in this embodiment, the clip 132h does not overlap with the pair of sensing units 131h and/or the communication unit 133h in the longitudinal direction of sensor 13h.

FIG. 12 illustrates a cardiac implant device 10i according to another embodiment of the present disclosure. Referring to FIG. 12, cardiac implant device 10i comprises a fixation member 11i, a sensor 13i, and a plurality of shunt members 12i. Sensor 13i and the plurality of shunt members 12i are configured to be attached to fixation member 11i. It should be noted that while cardiac implant device 10i comprises only two shunt members 12i in FIG. 12, in other embodiments, cardiac implant device 10i may comprise more shunt members 12i.

After the shunt device is implanted, the patient will usually receive other treatment, such as taking medication. After a period of time, the condition of some patients will improve. In this case, the shunt device will no longer match the patient's condition. Compared with a shunt channel with a larger cross-sectional area, a shunt channel with a smaller cross-sectional area is more likely to have tissue growth. The cardiac implant device 10i provided by the present disclosure comprises multiple shunt members 12i, that is, the cardiac implant device 10i provided by the present disclosure has multiple shunt channels. The multiple shunt members 12i can be integrally formed, that is, the shunt members in the present disclosure are not limited to independent components, but can also be a partial structure that forms a shunt channel. In this way, on the basis that the sum of the cross-sectional areas of the multiple shunt channels matches the patient's condition, the cross-sectional area of a single shunt channel can be relatively small, so that tissue growth is more likely to occur in a single shunt channel. When first implanted in the patient's heart, the sum of the cross-sectional areas of the multiple shunt channels meets the patient's condition at that time. After a period of usage, the patient's condition improves, and each shunt channel will experience a certain degree of tissue growth. Consequently, the sum of the cross-sectional areas of the multiple shunt channels will decrease fairly rapidly, and the sum of the cross-sectional areas of the multiple shunt channels will better match the patient's current condition. In contrast, if a cardiac implant device only comprises a single shunt member, the cross-sectional area of the shunt channel in the shunt member will be larger, making tissue growth less likely to occur within the shunt channel, or the rate of tissue growth will be slower. Thus, after a period of usage, the cardiac implant device will no longer be suitable for the patient's condition.

Continuing with FIG. 12, the plurality of shunt members 12i may comprise a first shunt member 12i-1 and a second shunt member 12i-2. The shunt channel of the second shunt member 12i-2 is constructed to be more likely to cause to tissue growth than the shunt channel of the first shunt member 12i-1. Thus, after a period of usage, the shunt channel of the first shunt member 12i-1 will have less tissue growth, and its shunt channel will maintain connectivity between the left and right atria, while the shunt channel of the second shunt member 12i-2 will have more tissue growth, and its shunt channel will become narrower or completely blocked, thereby reducing the overall shunt effect of the cardiac implant device 12i. Therefore, this structure is conducive to better matching the progression of the patient's condition.

There are many ways to achieve that the shunt channel of the second shunt member 12i-2 is more likely to have tissue growth than the shunt channel of the first shunt member 12i-1, which is not specifically limited in this disclosure. Several possible embodiments are given below.

In one embodiment, the inner wall of the shunt channel of the second shunt member 12i-2 may be rougher than the inner wall of the shunt channel of the first shunt member 12i-1. In this way, the shunt channel of the second shunt member 12i-2 will be more likely to cause tissue growth than the shunt channel of the first shunt member 12i-1. In another embodiment, the inner wall of the shunt channel of the second shunt member 12i-2 may be provided with a plurality of micropores (not shown in the figure) to cause tissue growth. In this way, the shunt channel of the second shunt member 12i-2 will be more likely to cause tissue growth than the shunt channel of the first shunt member 12i-1. In another embodiment, the plurality of micropores is filled with a filler that can be degraded and can cause tissue growth. For example, the filler can be, but is not limited to, selected from one or more of polyglycolic acid (PGA), polylactic acid (PLA), polylactic-glycolic acid copolymer (PLGA), polycaprolactone (PCL) or polyethylene glycol (PEG). Through this embodiment, the shunt channel of the second shunt member 12i-2 will be more likely to cause tissue growth than the shunt channel of the first shunt member 12i-1. In another embodiment, the inner wall of the shunt channel of the second shunt member 12i-2 can be provided with a coating that can be degraded and can cause tissue growth. For example, the coating can be, but is not limited to, selected from one or more of polyglycolic acid (PGA), polylactic acid (PLA), polylactic-glycolic acid copolymer (PLGA), polycaprolactone (PCL) or polyethylene glycol (PEG). In this way, the shunt channel of the second shunt member 12i-2 will be more likely to cause tissue growth than the shunt channel of the first shunt member 12i-1.

Of course, in addition to the above-mentioned possible embodiments, other methods can also be used to achieve the purpose of making the shunt channel of the second shunt member more likely to cause tissue growth than the shunt channel of the first shunt member.

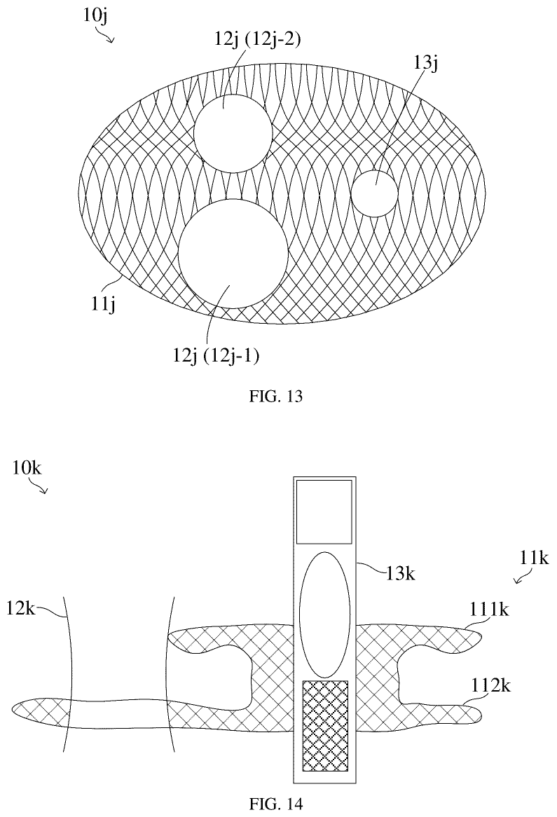

FIG. 13 illustrates a cardiac implant device 10j according to another embodiment of the present disclosure. Referring to FIG. 13, cardiac implant device 10j comprises a fixation member 11j, a sensor 13j, and multiple shunt members 12j. Sensor 13j and multiple shunt members 12j are configured to be attached to fixation member 11j. It should be noted that while FIG. 13 shows cardiac implant device 10j including only two shunt members 12j, in other embodiments, cardiac implant device 10j may comprise more shunt members 12j. The multiple shunt members 12j comprise a first shunt member 12j-1 and a second shunt member 12j-2. The cross-sectional area of the shunt channel of first shunt member 12j-1 is greater than the cross-sectional area of the shunt channel of second shunt member 12j-2. For example, the cross-sectional area of the shunt channel of first shunt member 12j-1 may be approximately 20 mm². It should be noted that, in the present disclosure, the cross-sectional area of a certain flow channel may refer to its minimum cross-sectional area or its average cross-sectional area.

Because the cross-sectional area of the shunt channel of the first shunt member 12j-1 is relatively large, tissue growth in the shunt channel is minimal after a period of usage, maintaining good connectivity between the left and right atria. Because the cross-sectional area of the shunt channel of the second shunt member 12j-2 is relatively small, tissue growth in the shunt channel is significant after a period of usage, causing the shunt channel to become narrower or completely blocked, thereby reducing the overall shunt effect of the cardiac implant device and better adapting to the patient's condition.

Continuing to refer to FIG. 13, the cross-sectional area of the shunt channel of the second shunt member 12j-2 can be less than or equal to 7 mm². The applicant found via research that when a child has a congenital atrial septal defect of less than or equal to 7 mm², the defect can be self-repaired after a period of time. Therefore, if the cross-sectional area of the shunt channel is less than or equal to 7 mm², after a period of usage, the shunt channel will be blocked due to tissue growth and other reasons, thereby reducing the overall shunt effect of the cardiac implant device 10j, thereby better matching the development of the patient's condition.

The fixation member 11j and the shunt member 12j can be integrally formed. For example, by braiding elastic wires or cutting elastic metals, or they can be formed separately and then connected by welding or other processes.

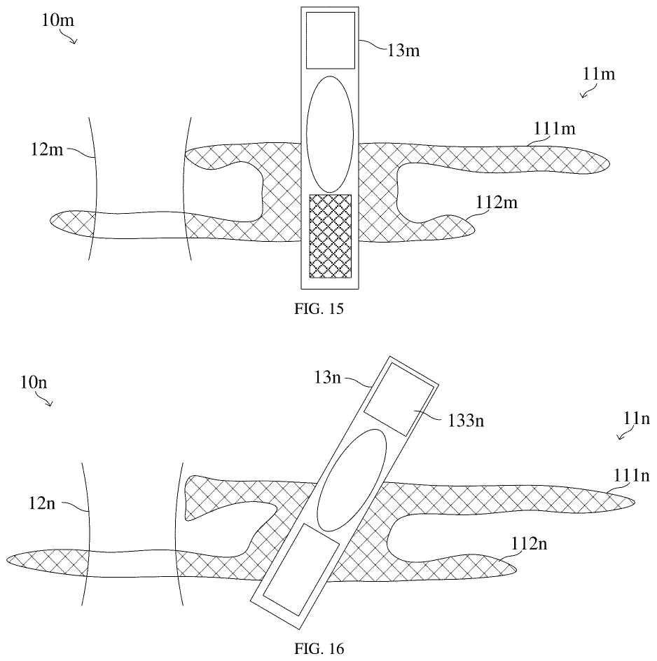

FIG. 14 illustrates a cardiac implant device 10k according to another embodiment of the present disclosure. Referring to FIG. 14, the cardiac implant device 10k comprises a fixation member 11k, a shunt member 12k, and a sensor 13k. The shunt member 12k and the sensor 13k are configured to be attached to the fixation member 11k. The fixation member 11k comprises a first fixing disc 111k and a second fixing disc 112k, with the first fixing disc 111k being smaller than the second fixing disc 112k. The shunt member 12k is attached to the second fixing disc 112k. This configuration ensures that the cardiac implant device 10k is securely fixed to the atrial septum of the patient's atrium while maintaining a relatively small overall size. FIG. 15 illustrates a cardiac implant device 10m according to another embodiment of the present disclosure. Referring to FIG. 15, the cardiac implant device 10m comprises a fixation member 11m, a shunt member 12m, and a sensor 13m. The shunt member 12m and sensor 13m are configured to be attached to the fixation member 11m. The fixation member 11m comprises a first fixing disc 111m and a second fixing disc 112m, which are arranged in an offset manner. The shunt member 12m is configured to be attached to the second fixing disc 112m. This configuration facilitates more securely securing the cardiac implant device 10m to the atrial septum of a patient's atrium.

FIG. 16 illustrates a cardiac implant device 10n according to another embodiment of the present disclosure. Referring to FIG. 16, cardiac implant device 10n comprises a fixation member 11n, a shunt member 12n, and a sensor 13n. Sensor 13n comprises a communication unit 133n for communicating with an external device. Fixation member 11n comprises a pair of fixing discs 111n, 112n (also referred to as fixing parts 111n, 112n). The sensing unit is configured to be tilted relative to the pair of fixing discs 111n, 112n, such that communication unit 133n is positioned away from shunt member 12n. This configuration avoids or minimizes the impact of shunt member 12n on the quality of communication between communication unit 133n and the external device.

There are many ways to implement a shunt member. The present disclosure does not specifically limit the specific embodiment of the shunt member. In certain embodiments, the shunt member may comprise a stent and a covering membrane. The covering membrane is attached to the stent, and the stent is attached to the fixation member. The covering membrane can be attached to the inner circumferential side of the stent or to the outer circumferential side of the stent. There are also many ways to implement a stent. For example, the stent can be a self-expanding stent. For another embodiment, the stent can also be an expandable stent. Below, an exemplary stent is given in conjunction with the accompanying drawings.

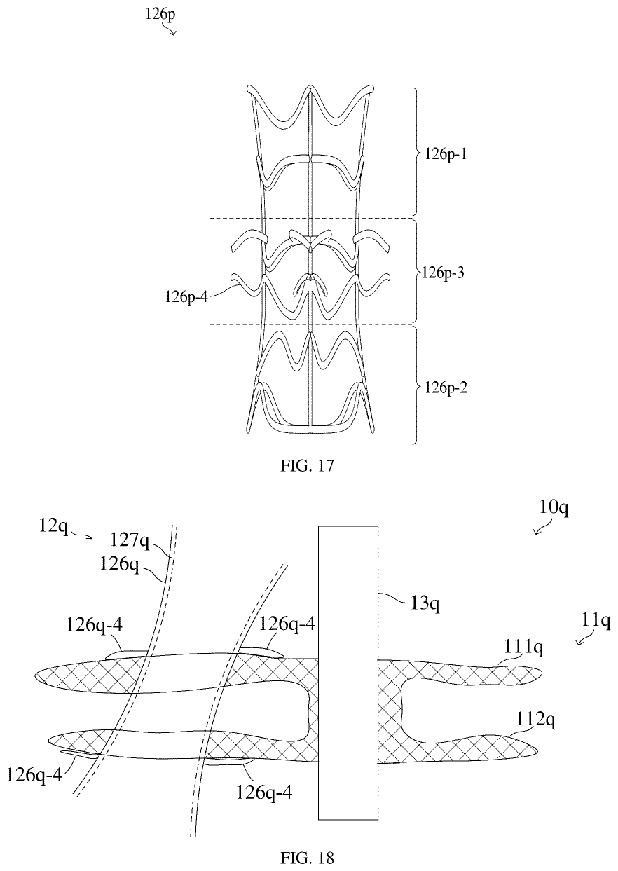

FIG. 17 shows a stent 126p according to an embodiment of the present disclosure. Referring to FIG. 17, stent 126p comprises a pair of flared portions 126p-1 and 126p-2 and a neck portion 126p-3 located therebetween. The flared portions 126p-1 and 126p-2 are configured to be self-expanding, and the neck portion 126p-3 is configured to be expandable.

The fixation member may be provided with a mounting hole for mounting the stent 126p. During the installation of the stent 126p to the fixation member, the pair of flared portions 126p-1 and 126p-2 may be maintained in a collapsed state while the stent 126p is inserted into the mounting hole. Subsequently, the pair of flared portions 126p-1 and 126p-2 may expand so that the flared portions 126p-1 and 126p-2 are located on opposite sides of the fixation member. Subsequently, the neck portion 126p-3 may be expanded using an operation device (e.g., a balloon) to match the size of the mounting hole or the patient's condition. This embodiment allows the stent 126p to be relatively conveniently and reliably secured to the fixation member. In addition, since the neck portion 126p is constructed to be expandable, during the implantation process, the inner diameter of the stent 126p can be adjusted by an operation device (e.g., a balloon), thereby adjusting the inner diameter of the shunt channel (i.e., the inner diameter of the shunt channel at the neck portion), thereby making the inner diameter of the shunt channel better match the patient's condition.

Continue with FIG. 17, the stent 126p is provided with a plurality of first gripping elements 126p-4 on its outer circumferential side. The stent 126p is configured to be attached to the fixation member via the plurality of first gripping elements 126p-4. The plurality of first gripping elements 126p-4 allows the stent 126p to be more securely fixed to the fixation member.

FIG. 18 illustrates a cardiac implant device 10q according to another embodiment of the present disclosure. Referring to FIG. 18, cardiac implant device 10q comprises a fixation member 11q, a shunt member 12q, and a sensor 13q. The shunt member 12q and sensor 13q are configured to be attached to the fixation member 11q. The fixation member 11q comprises a first fixing disc 111q and a second fixing disc 112q. The shunt member 12q comprises a stent 126q and a covering membrane 127q. The stent 126q comprises a plurality of first gripping elements 126q-4. The plurality of first gripping elements 126q-4 comprises two groups of first gripping elements 126q-4 distributed longitudinally. Each group of first gripping elements 126q-4 may comprise a plurality of first gripping elements 126q-4 arranged circumferentially. As shown in FIG. 18, one group of first gripping elements 126q-4 is located on a side of the first fixing disc 111q away from the second fixing disc 112q, and another group of first gripping elements 126q-4 is located on a side of the second fixing disc 112q away from the first fixing disc 111q.

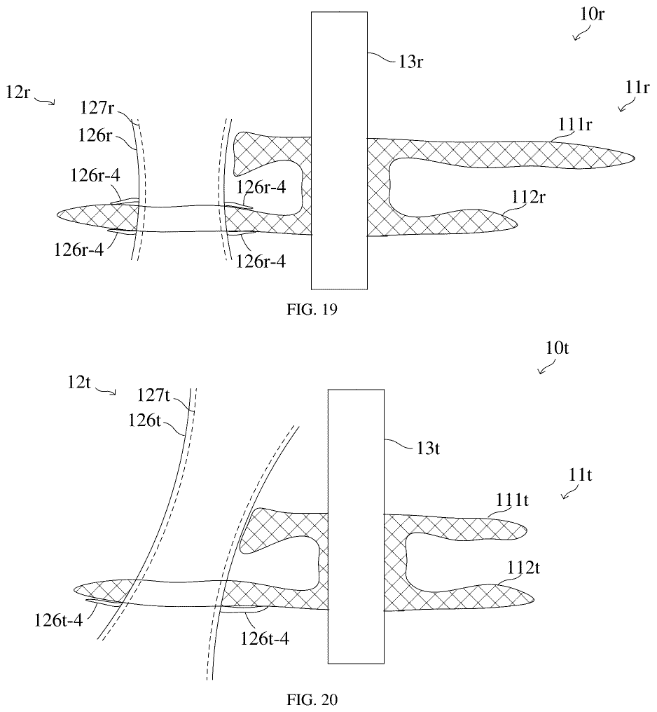

FIG. 19 illustrates a cardiac implant device 10r according to another embodiment of the present disclosure. Referring to FIG. 19, cardiac implant device 10r comprises a fixation member 11r, a shunt member 12r, and a sensor 13r. The shunt member 12r and sensor 13r are configured to be attached to the fixation member 11r. The fixation member 11r comprises a first fixing disc 111r and a second fixing disc 112r. The shunt member 12r is configured to be fixed to the second fixing disc 112r. The shunt member 12r comprises a stent 126r and a covering membrane 127r. The stent 126r comprises a plurality of first gripping elements 126r-4. The plurality of first gripping elements 126r-4 comprise two groups of first gripping elements 126r-4 distributed longitudinally. Each group of first gripping elements 126r-4 may comprise a plurality of first gripping elements 126r-4 arranged circumferentially. As shown in FIG. 19, one group of first gripping elements 126r-4 is located on a side of the second fixing disc 112r facing away from the first fixing disc 111r, and another group of first gripping elements 126r-4 is located on a side of the second fixing disc 112r facing the first fixing disc 111r.

FIG. 20 illustrates a cardiac implant device 10t according to another embodiment of the present disclosure. Referring to FIG. 18, the cardiac implant device 10t comprises a fixation member 11t, a shunt member 12t, and a sensor 13t. The shunt member 12t and the sensor 13t are configured to be attached to the fixation member 11t. The fixation member 11t comprises a first fixing disc 111t and a second fixing disc 112t. The shunt member 12t comprises a stent 126t and a covering membrane 127t. The stent 126t comprises a plurality of first gripping elements 126t-4. The plurality of first gripping elements 126t-4 are arranged circumferentially. Referring to FIG. 20, the plurality of first gripping elements 126t-4 are located on a side of the second fixing disc 112t facing away from the first fixing disc 111t.

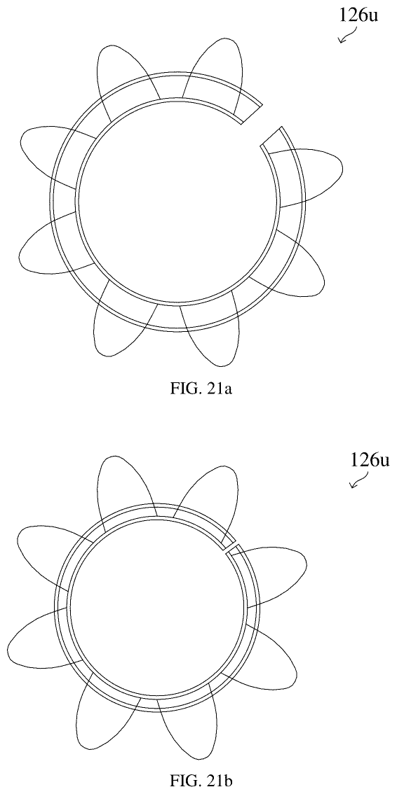

FIGS. 21a and 21b illustrate schematic cross-sectional views of a stent 126u according to an embodiment of the present disclosure. Referring to FIGS. 21a and 21b, the cross-section of stent 126u is shaped in an open ring. This allows the inner diameter of stent 126u, and thus the inner diameter of the shunt channel, to be adjusted during implantation using an operation device (e.g., a balloon).

There are various ways to attach the sensor to the fixation member. This disclosure does not specifically limit how to attach the sensor to the fixation member. A possible embodiment is described below with reference to the accompanying drawings.

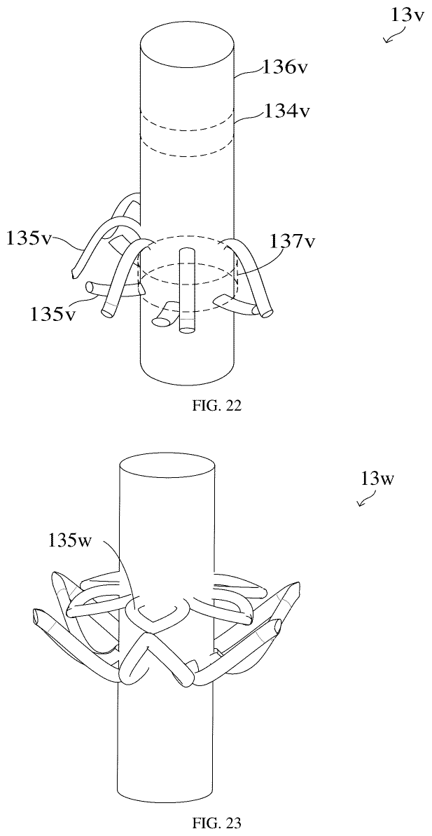

FIG. 22 shows a sensor 13v according to an embodiment of the present disclosure. Referring to FIG. 22, the outer periphery of the sensor 13v is provided with a plurality of second gripping elements 135v, and the sensor 13v is attached to the fixation member via the plurality of second gripping elements 135v. The plurality of second gripping elements 135v allows the sensor 13v to be securely fixed to the fixation member.

Continuing with FIG. 22, the plurality of second gripping elements 135v comprise two groups of second gripping elements 135v spaced apart longitudinally along the sensor 13v. Each group of second gripping elements 135v is arranged circumferentially around the sensor 13v. The two groups of second gripping elements 135v are curved toward each other. This configuration allows the sensor 13v to be more reliably secured to the fixation member. The second gripping elements 135v can also be integrally formed with the fixation member.

Continuing with reference to FIG. 22, the sensor 13v comprises a cylindrical main body 136v and a sleeve 137v sleeved on the main body 136v. A plurality of second gripping elements 135v are attached to the main body 136v via the sleeve 137v. Specifically, each second gripping element 135v can be partially inserted between the sleeve 137v and the main body 136v to be fixed to the main body 136v. In this way, the plurality of second gripping elements 135v can be fixed to the main body 136v of the sensor. It should be understood that in other embodiments, the plurality of second gripping elements 135v can also be fixed to the main body 136v by other means, For example, by welding or bonding.

Continuing with reference to FIG. 22, the sleeve 137v can be constructed to be slidable in the longitudinal direction of the main body 136v. In this way, during the implantation process, the physician can adjust the position of the main body 136v of the sensor 13v relative to the fixation member according to actual conditions. For example, the position of the main body 136v can be adjusted to ensure that the sensing unit is directly exposed to the blood in the atrium, or the position of the main body can be adjusted to ensure that the signal of the communication unit 134v is not excessively blocked. The multiple second gripping elements 137v do not overlap with the communication unit 134v in the longitudinal direction of the sensor 13v. According to this construction, the multiple second gripping elements 137v will not or minimally affect the communication quality between the communication unit 134v and the external device.

Continuing with reference to FIG. 22, each second gripping element 137v has an open-ring shape, which helps reduce the overall weight and size of the plurality of gripping elements 137v, thereby contributing to miniaturization and lightweight of the cardiac implant device.

FIG. 23 illustrates a sensor 13w according to an embodiment of the present disclosure. Sensor 13w is substantially identical to sensor 13v, and for the sake of brevity, the similarities are not further described. Referring to FIG. 23, the second gripping element 135w of sensor 13w has a closed-ring shape. This structure allows sensor 13w to be more reliably secured to the fixation member.

According to the cardiac implant device provided herein, the sensor can be configured to be located between the shunt member and the patient's anterior chest wall. Accordingly, the external device communicating with the sensor's communication unit is configured to be located outside the patient's anterior chest wall. This prevents the shunt member from being located between the sensor and the external device, and thus avoids or minimizes the impact of the shunt member on the communication quality between the sensor's communication unit and the external device.

Alternatively, according to the cardiac implant device provided by the present disclosure, the sensor may be constructed to be located between the shunt member and the patient's left chest wall. Correspondingly, the external device that communicates with the communication unit of the sensor is configured to be outside the patient's left chest wall. In this way, the shunt member will not be located between the sensor and the external device, and therefore, the shunt will not or only minimally affect the communication quality between the communication unit of the sensor and the external device. Alternatively, according to the cardiac implant device provided by the present disclosure, the sensor may be constructed to be located between the shunt member and the patient's posterior chest wall. Correspondingly, the external device that communicates with the communication unit of the sensor is configured to be outside the patient's posterior chest wall. In this way, the shunt will not be located between the sensor and the external device, and therefore, the shunt will not or less affect the communication quality between the communication unit of the sensor and the external device.

In variations of the above-described embodiments, the fixing disc may also have a fixing part or fixing parts in other shapes, such as the claw-shaped, open-ring shape of the second gripping element 135v shown in FIG. 22, or the claw-shaped, closed-ring shape of the second gripping element 135w shown in FIG. 23, or the fixing part in its deployed status may have at least a portion that is parallel to the atrial septum. As long as the fixing part can secure the fixation member to the atrial septum. In variations of the above-described embodiments, the shunt member may be integrally woven with the fixation member, and this is not a limitation of the present invention.

It should be understood that the term “comprising” and its variations used in this disclosure are open-ended, i.e., “comprising but not limited to”. The term “according to” means “at least in part according to”. The term “one embodiment” means “a pair of embodiments”, and the term “another embodiment” means “a pair of additional embodiments”. It should be understood that although the terms “first” or “second” etc., may be used in this disclosure to describe various elements (such as the first gripping element and the second gripping element of a shunt member), these elements are not limited by these terms; these terms are merely used to distinguish one element from another.

The above description is merely specific embodiments of the present disclosure, but the scope of protection of the present disclosure is not limited thereto. Any changes or substitutions that can be easily conceived by a person skilled in the art within the technical scope disclosed in this disclosure should be comprised in the scope of protection of the present disclosure. Therefore, the scope of protection of the present disclosure should be based on the scope of protection of the claims.

1. A cardiac implant device, characterized by comprising:
a fixation member, having a collapsed state for being capable of passing through a created opening on an atrial septum and an expanded state for fixing the fixation member on the atrial septum; and
a shunt member, configured to be attached to the fixation member and capable of passing through the created opening, wherein the shunt member is configured to define a shunt channel that passes through the created opening and connects a left atrium and a right atrium of a patient.

2. The cardiac implant device according to claim 1, characterized in that
the fixation member comprises a pair of fixing parts that can be fixed to the atrial septum; and each of the fixing parts is a fixing disc, and the shunt channel has a first opening configured to connect to the left atrium and a second opening configured to connect to the right atrium respectively, and the first opening and the second opening are configured such that: a distance between orthographic projections of the first opening and the second opening on a projection plane substantially parallel to the pair of fixing parts ranges from 10 mm to 30 mm, so that when the patient is in at least one posture, the first opening is higher than the second opening in a direction of gravity.

3. The cardiac implant device according to claim 2, characterized in that
the first opening and the second opening are configured such that the orthographic projection of the first opening on the projection plane is located further anterior and/or further superior than the orthographic projection of the second opening on the projection plane.

4. The cardiac implant device according to claim 2, characterized in that
the shunt member is constructed in a straight cylindrical shape and is inclined relative to the projection plane; or, the shunt member is constructed in an S-shape.

5. The cardiac implant device according to claim 1, characterized in that
the cardiac implant device comprises a plurality of shunt members, wherein the plurality of shunt members comprises a first shunt member and a second shunt member, and the shunt channel of the second shunt member is constructed to be more likely to cause tissue growth than the shunt channel of the first shunt member.

6. The cardiac implant device according to claim 5, characterized in that
an inner diameter of the shunt channel of the first shunt member is larger than an inner diameter of the shunt channel of the second shunt member, and
the cross-sectional area of the shunt channel of the second shunt member is less than or equal to 7 mm².

7. (canceled)

8. The cardiac implant device according to claim 5, characterized in that
an inner wall of the shunt channel of the second shunt member is rougher than an inner wall of the shunt channel of the first shunt member.

9. The cardiac implant device according to claim 5, characterized in that
an inner wall of the shunt channel of the second shunt member is provided with a plurality of micropores to cause tissue growth, and the plurality of micropores are filled with fillers that can be degraded and is capable of causing tissue growth.

10. The cardiac implant device according to claim 5, characterized in that
an inner wall of the shunt channel of the second shunt member is provided with a coating that is degradable and capable of causing tissue growth.

11. The cardiac implant device according to claim 1, characterized in that
the shunt member comprises a stent and a covering membrane located on an inner circumferential side or an outer circumferential side of the stent, wherein the stent comprises a pair of flared portions and a neck portion located therebetween, wherein the pair of flared portions are constructed to be self-expanding, and the neck portion is constructed to be expandable, wherein a cross-section of the stent is shaped in an open ring.

12. The cardiac implant device according to claim 11, characterized in that
the stent is provided with a plurality of first gripping elements on its outer circumferential side, and the stent is configured to be attached to the fixation member through the plurality of first gripping elements.

13. The cardiac implant device according to claim 1, characterized in that
the cardiac implant device further comprises a sensor configured to be attached to the fixation member to sense physiological information of the patient.

14. The cardiac implant device according to claim 13, characterized in that
the fixation member comprises a pair of fixing parts that can be fixed to the atrial septum and a waist part located between the pair of fixing parts and constructed to pass through the created opening, wherein one of the shunt member and the sensor passes through the waist part or is connected to the waist part, and the other does not pass through the waist part; and the fixing part is a fixing disc.

15. The cardiac implant device according to claim 13, characterized in that
the fixation member comprises a pair of fixing parts that can be fixed to the atrial septum and a waist part located between the pair of fixing parts and constructed to pass through the created opening, wherein the shunt member and the sensor both pass through the waist part; and
each fixing part is a fixing disc, and each fixing disc is elliptical with a pair of focal points, and the shunt member and the sensor are respectively located at the pair of focal points.

16. The cardiac implant device according to claim 13, characterized in that
the sensor is constructed to pass through the created opening, and at least one end of the sensor is provided with a sensing unit that is at least partially exposed outside the fixation member.

17. The cardiac implant device according to claim 13, characterized in that
the fixation member comprises a pair of fixing parts that can be fixed to the atrial septum; and each fixing part is a fixing disc, and the pair of fixing parts comprises a first fixing disc and a second fixing disc, wherein the first fixing disc is fixed with a retaining ring, and the sensor is fixed to the second fixing disc and is provided with one or more clips adapted to the retaining ring, and the sensor also comprises a communication unit for communicating with an external device, wherein the one or more clips and the communication unit at least partially do not overlap in the longitudinal direction of the sensor, and at least a portion of the retaining ring is made of a radiopaque material.

18. The cardiac implant device according to claim 13, characterized in that
a plurality of second gripping elements are provided on an outer periphery of the sensor, and the sensor is attached to the fixation member via the second gripping elements, and each second gripping element has an open-ring shape or a closed-ring shape and the plurality of second gripping elements comprise two groups of second gripping elements spaced apart in a longitudinal direction of the sensor, and the two groups of second gripping elements are bent toward each other.

19. The cardiac implant device according to claim 18, characterized in that
the sensor comprises a cylindrical main body and a sleeve sleeved on the main body, wherein the plurality of second gripping elements is attached to the main body via the sleeve; and the sleeve is configured to be slidable along the longitudinal direction of the main body.

20. The cardiac implant device according to claim 18, characterized in that
the sensor further comprises a communication unit for communicating with an external device, wherein the plurality of second gripping elements and the communication unit do not overlap in the longitudinal direction of the sensor.

21. The cardiac implant device according to claim 13, characterized in that
the fixation member comprises a pair of fixing parts that can be fixed to the atrial septum; and the sensor also comprises a communication unit for communicating with an external device, wherein the sensor is constructed to be inclined relative to the pair of fixing parts so that the communication unit is away from the shunt member; and each fixing part is a fixing disc.

22. The cardiac implant device according to claim 13, characterized in that
the sensor is constructed to be located between the shunt member and an anterior chest wall of the patient; or, the sensor is constructed to be located between the shunt member and a left chest wall of the patient; or, the sensor is constructed to be located between the shunt member and a posterior chest wall of the patient.

23. The cardiac implant device according to claim 1, characterized in that
the fixation member comprises a pair of fixing parts that can be fixed to the atrial septum; and the pair of fixing parts are distributed in an offset manner, and the shunt member is constructed to be attached to one of the fixing parts; and each fixing part is a fixing disc.

24. The cardiac implant device according to claim 1, characterized in that
the fixation member comprises a pair of fixing parts that can be fixed to the atrial septum; and the pair of fixing parts comprise a first fixing disc and a second fixing disc, wherein the first fixing disc is larger than the second fixing disc, and the shunt member is attached to the first fixing disc.

25. The cardiac implant device according to claim 13, characterized in that
the fixation member comprises a pair of fixing parts that can be fixed to the atrial septum; the fixation member is made by braiding strands of wire radially so that a braiding density of each fixing part gradually decreases from a position where the sensor is located to an edge of each fixing part.
